SEQUENCE LISTING (8 sequences)

SEQ ID NO: 1 - DNA, 20 bp, synthetic construct
  misc_feature 1..20: note = Synthetic: P5
  source 1..20: mol_type = other DNA
aatgatacggcgaccaccga

SEQ ID NO: 2 - DNA, 21 bp, synthetic construct
  misc_feature 1..21: note = Synthetic: P7
  source 1..21: mol_type = other DNA
caagcagaagacggcatacga

SEQ ID NO: 3 - DNA, 20 bp, synthetic construct
  misc_feature 1..20: note = Synthetic: anti-P5
  source 1..20: mol_type = other DNA
tcggtggtcgccgtatcatt

SEQ ID NO: 4 - DNA, 21 bp, synthetic construct
  misc_feature 1..21: note = Synthetic: anti-P7
  source 1..21: mol_type = other DNA
tcgtatgccgtcttctgcttg

SEQ ID NO: 5 - DNA, 33 bp, synthetic construct
  misc_feature 1..33: note = Synthetic: SBS3
  source 1..33: mol_type = other DNA
acactctttccctacacgacgctcttccgatct

SEQ ID NO: 6 - DNA, 37 bp, synthetic construct
  misc_feature 1..37: note = Synthetic: SBS8
  source 1..37: mol_type = other DNA
cggtctcggcattcctgctgaaccgctcttccgatct

SEQ ID NO: 7 - DNA, 33 bp, synthetic construct
  misc_feature 1..33: note = Synthetic: anti-SBS3
  source 1..33: mol_type = other DNA
agatcggaagagcgtcgtgtagggaaagagtgt

SEQ ID NO: 8 - DNA, 37 bp, synthetic construct
  misc_feature 1..37: note = Synthetic: anti-SBS8
  source 1..37: mol_type = other DNA
agatcggaagagcggttcagcaggaatgccgagaccg

(19) United States
(12) Patent Application Publication
(10) Pub. No.: US 2026/0022495 A1
(43) Pub. Date: Jan. 22, 2026

(54) APTAMER DISCOVERY AND SELECTION TECHNIQUES

(71) Applicant: ILLUMINA, INC., San Diego, CA (US)

(72) Inventors: Miguel Angel Aleman Garcia, San Diego, CA (US); Alexandre Richez, San Diego, CA (US)

(21) Appl. No.: 18/876,788

(22) PCT Filed: Apr. 15, 2024
(86) PCT No.: PCT/US2024/024626

§ 371 Date: Dec. 19, 2024

Related U.S. Application Data
(60) Provisional application No. 63/495,937, filed on Apr. 13, 2023.

(51) Int. Cl. C40B 40/06; C12N 15/113; C12Q 1/6811

(52) U.S. Cl. CPC C40B 40/06; C12N 15/113; C12Q 1/6811; C12N 2310/16; C12N 2320/13; C12N 2330/31

(57) ABSTRACT

Encoded aptamer candidate libraries with nucleotide modification are described. The aptamer candidates include a first conserved primer region and a second conserved primer region. aptamer candidates also include a variable region disposed between the first conserved primer region and the second conserved primer region and that includes at least one modified nucleotide. A code region includes a nucleotide sequence that is unique for a modification type of the at least one modified nucleotide such that a sequence of each aptamer candidate can be used to identify the associated modification type.

### CROSS-REFERENCE TO RELATED APPLICATIONS

This application is a national stage application claiming priority to PCT/US24/24626, which claims priority to and the benefit of U.S. Provisional Application No. 63/495,937, entitled “APTAMER DISCOVERY AND SELECTION TECHNIQUES” and filed on Apr. 13, 2023, the disclosures of which are hereby incorporated by reference in their entireties.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 30, 2023, is named “IP-2332-PRV.xml” and is 11,047 bytes in size. The sequence listing contained in this .XML file is part of the specification and is hereby incorporated by reference herein in its entirety.

### BACKGROUND

The disclosed technology relates generally to aptamer discovery, generation, and/or selection techniques. In particular, the technology disclosed relates to techniques for improved, and more diverse, aptamer candidates for use in aptamer libraries to select aptamers for aptamer-based assays or for therapeutic applications.

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Protein expression patterns help define a cell's identity and state. RNA transcripts are often used as a surrogate for protein expression, but the relationship between abundance of proteins and mRNA is not one-to-one. There are differences caused by regulation of posttranscriptional, translational and protein degradation. Therefore, direct nucleic acid sequencing of RNA transcripts may not provide an accurate estimation of protein expression.

Aptamers are nucleic acids that bind to molecular targets, such as proteins, with high affinity and specificity. Advancements in aptamer selection and design include Systematic Evolution of Ligands by EXponential enrichment (SELEX). In SELEX, high affinity nucleic acids for different analytes of interest can be isolated from a combinatorial library, permitting high throughput characterization of aptamer-target binding and multiplexed assays for analytes in a complex biological sample. Upon aptamer binding to an analyte target, the binding event can be detected to characterize the presence and concentration of various analytes in the biological sample. However, the available panel of aptamers for use in protein detection is a function of the aptamer discovery process, and not all desired protein targets may yield an aptamer with appropriate binding characteristics. Therefore, improved aptamer generation and discovery would be beneficial for expanding available protein targets.

### BRIEF DESCRIPTION

In one embodiment, the present disclosure provides an aptamer candidate library. The library includes a plurality of at least partially single-stranded nucleic acids. An individual at least partially single-stranded nucleic acid of the plurality includes a first conserved primer region that is conserved among the plurality of at least partially single-stranded nucleic acids; a second conserved primer region that is conserved among the plurality of at least partially single-stranded nucleic acids; a variable region disposed between the first conserved primer region and the second conserved primer region, wherein the variable region is variable among the plurality of at least partially single-stranded nucleic acids and wherein each variable region comprises at least one modified nucleotide; and a code region, wherein the code region comprises a nucleotide sequence that is unique for a modification type of the at least one modified nucleotide

In one embodiment, the present disclosure provides aptamer selection method that includes providing a library of aptamer candidates of different aptamer subgroups, wherein each aptamer candidate of an individual subgroup of the different aptamer subgroups comprises: a first conserved primer region that is conserved among the aptamer candidates; a second conserved primer region that is conserved among the aptamer candidates; a variable region disposed between the first conserved primer region and the second conserved primer region, wherein the variable region is variable among the aptamer candidates and wherein the variable region comprises at least one modified nucleotide; and a code region, wherein the code region comprises a nucleotide sequence that is unique for a modification type of the at least one modified nucleotide and that uniquely identifies the modification type from different modification types of other subgroups of the different subgroups. The method also includes selecting an aptamer candidate based on binding to a target molecule; amplifying the selected aptamer candidate using primers based on the first and second conserved primer region; and

sequencing the amplified aptamer candidate to determine a sequence of the variable region and to identify the modification type.

In one embodiment, the present disclosure provides aptamer candidate library that includes a plurality of partial duplex aptamer candidates comprising a first strand and a second strand. The first strand includes a duplex portion comprising a first complementary region that hybridizes to a second complementary region of the second strand; a single-stranded portion, the single-stranded portion comprising: a first conserved primer region that is conserved among first strands and second strands of the plurality of partial duplex aptamer candidates; a second conserved primer region that is conserved among the first strands and the second strands of the plurality of partial duplex aptamer candidates; a variable region disposed between the first conserved primer region and the second conserved primer region, wherein the variable region is variable among the first strands and the second strands of the plurality of partial duplex aptamer candidates and that comprises at least one modified nucleotide; and a code region, wherein the code region comprises a nucleotide sequence that is unique for a modification type of the at least one modified nucleotide.

The preceding description is presented to enable the making and use of the technology disclosed. Various modifications to the disclosed implementations will be apparent, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein. The scope of the technology disclosed is defined by the appended claims.

### BRIEF DESCRIPTION OF THE DRAWINGS

These and other features, aspects, and advantages of the disclosed embodiments will become better understood when the following detailed description is read with reference to the accompanying drawings in which like characters represent like parts throughout the drawings, wherein:

FIG. 1 is a schematic illustration of prior art SELEX approaches;

FIG. 2 is a schematic illustration of a SELEX approach for aptamer selection from a candidate library with multiple modifications, according to an embodiment of the disclosure;

FIG. 3 is a schematic illustration of amplification and characterization of a selected aptamer candidate, according to an embodiment of the disclosure;

FIG. 4 is a schematic illustration of a SELEX approach for aptamer selection that facilitates duplex formation, according to an embodiment of the disclosure;

FIG. 5 is a schematic illustration of amplification and characterization of a selected aptamer duplex candidate, according to an embodiment of the disclosure;

FIG. 6 shows example uridine modifications that may be incorporated into aptamers, according to an embodiment of the disclosure;

FIG. 7 is a schematic illustration of conserved and variable nucleotide regions in aptamer nucleotide sequences, according to an embodiment of the disclosure;

FIG. 8 is a schematic illustration of a SELEX selection cycle with aptamer amplification, according to an embodiment of the disclosure;

FIG. 9 is a schematic illustration of a SELEX selection cycle with aptamer amplification, according to an embodiment of the disclosure; and

FIG. 10 is a block diagram of a sequencing device configured to acquire sequencing data in accordance with the present techniques.

### DETAILED DESCRIPTION

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

Aptamers are short single stranded nucleic acid molecules (ssDNA or ssRNA) or modified nucleic acids that can bind to their specific target molecules with high affinity. In addition to use in high throughput multiomic applications, identification of aptamers with high specific binding for a molecule of interest can be used in therapeutics. For example a pegylated anti-vascular endothelial growth factor aptamer, pegaptanib, has been approved for clinical use. Aptamers are selected using a method known as Systematic Evolution of Ligands by EXponential enrichment (SELEX). In SELEX, a library of up to 10¹⁵ different candidate sequences (e.g., candidate aptamers) of nucleic acid molecules is synthesized. However, conventional SELEX does not always produce aptamers with the desired specificity and affinity to the target molecule.

The generation of high-quality aptamers for relevant targets remains a major challenge. One of the biggest limitations is the use of structures with restricted chemical diversity making the process of finding high affinity specific aptamers harder. Unlike peptides, single stranded DNA molecules do not have a wide range of chemical groups. The limited chemical diversity of the nucleotide-based library restricts finding successful aptamers. Aptamers generally have four nucleotide building block components. Further, oligonucleotides are negatively charged polymers, which also restricts the chemical characteristics of the candidate aptamers. In contrast, antibodies have the benefit of combining 20 amino acids in their sequence to reaching high degree of selectivity and sensitivity.

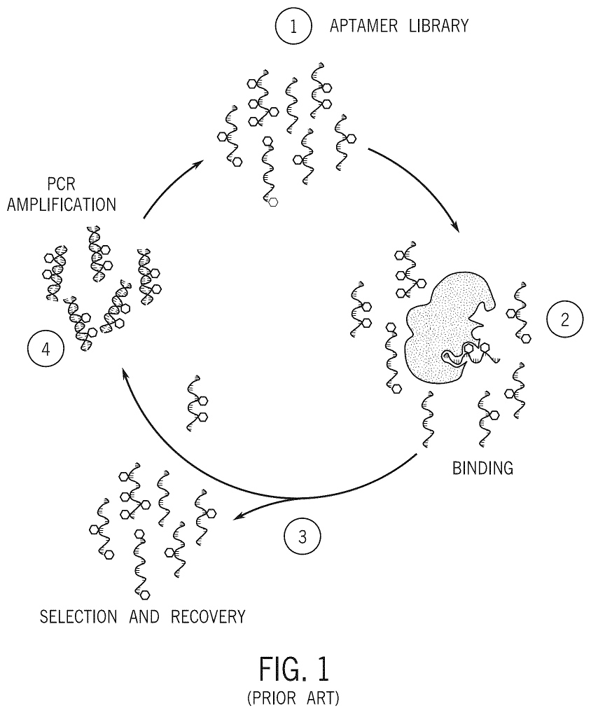

To overcome this limited range of starting materials, several strategies have been reported in the last years including, LOOPER-SELEX, Click-SELEX, X-SELEX, post-SELEX modifications and the use of modified nucleic bases in the SELEX step (i.e SOMAmers). These aptamers with expanded chemical diversity have shown enhanced binding properties in comparison with their unmodified version. FIG. 1 shows an example prior art approach for aptamer selection. The first step is to generate a library of single-stranded DNAs (ssDNAs). This can include ssDNAs with one modified nucleotide, typically dUTP, modified with an aromatic molecule. Most aptamers identified in this approach are not selective enough for a target protein and need to be further modified by help of computational and mutation studies. Thus, after selection of the best aptamer sequence, a second step involving computational modelling and the creation of a new library with new modifications on the selected sequence is performed. Screening the new library or successive new libraries is time consuming and inefficient, and often 5-15 rounds of selection may be involved. Accordingly, existing approaches to develop aptamers with high selectivity and affinity are not sufficient.

Provided herein are techniques for aptamer generation that integrate encoded small-organic molecules or other modifications in a single-stranded or partially single-stranded SELEX library. The combination of two molecular evolution-based technologies: aptamer discovery and DNA-Encoded Libraries facilitates the generation of aptamers with large chemical diversity, such as small molecule-DNA hybrid aptamers, overcoming the lack of chemical diversity in existing approaches and one of the biggest challenges in aptamer discovery. The generation of aptamer candidates with increased chemical diversity has the advantage of generating aptamers that are highly selective and specific for a given target molecule (i.e. proteins). The use of DNA encoding permits each nucleotide modification type to be identified via its associated encoded DNA sequence, such that an identified candidate aptamer can be characterized by both its binding sequence and its modification type and/or modification location. The disclosed aptamer discovery techniques that harness DNA encoded libraries in combination with the SELEX process may introduce up to millions of modified-fragments in a single selection round, overcoming the limitations of traditional aptamers and delivering the following advantages: high throughput screening, expanded chemical space, easy synthesis, stability, fast generation time, low cost of production and high specify and selectivity.

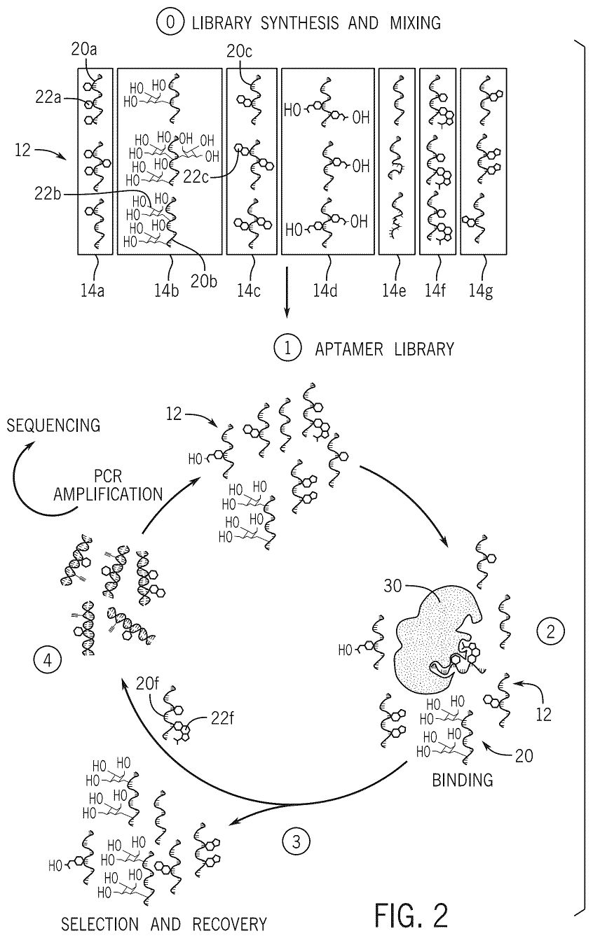

FIG. 2 shows an example workflow for encoded aptamer selection according to the present techniques. In the illustrated embodiment, an aptamer candidate library 12 is formed from combining different aptamer candidate subgroups 14 (illustrated as different subgroups 14a, 14b, 14c, 14d, 14c, 14f, 14g, and referred to collectively as aptamer candidate library 12), with different subgroups 14 having respective different types of modification/s of nucleotides relative to one another. It should be understood that the illustration is by way of example, and any number of different subgroups 14 may be used to form the aptamer candidate library 12. Each subgroup 14 can be formed separately, and the separate subgroup 14 then pooled with other subgroups 14 to generate the aptamer library 12. In this manner, the particular modification or group of modifications used to generate one subgroup 14 does not modify the other subgroups 14.

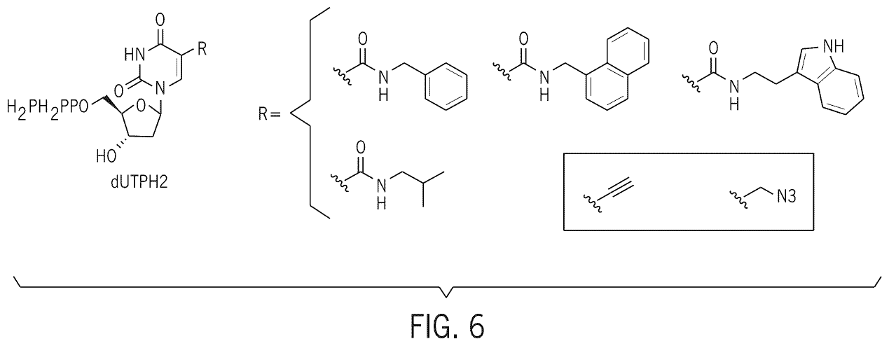

In one example, the different types of modifications of each subgroup 14 may include different types of base modifications and/or different identities of modified bases. For example, aptamer candidates 20a in the first subgroup 14a can be generated using a particular type of modified uridine (dUTP) 22a. Aptamer candidates 20b in the second subgroup 14b can be generated using a different type of modified uridine (dUTP) 22b such that the modified uridine 22a is chemically distinguishable from the modified uridine 22b. Aptamer candidates 20c in the third subgroup 14c can be generated using a modified adenosine 22c, and so on. The identity of the modified nucleotides 22 (A,C,T,U,G) can be changed or combined to increase the library diversity. An individual subgroup 14 of the library 12 can be formed using a single type of modified nucleotide 22 or two or more types of modified nucleotides 22. The types of chemical modifications may include those discussed herein (see FIG. 6), and may include modifications introduced via click chemistry.

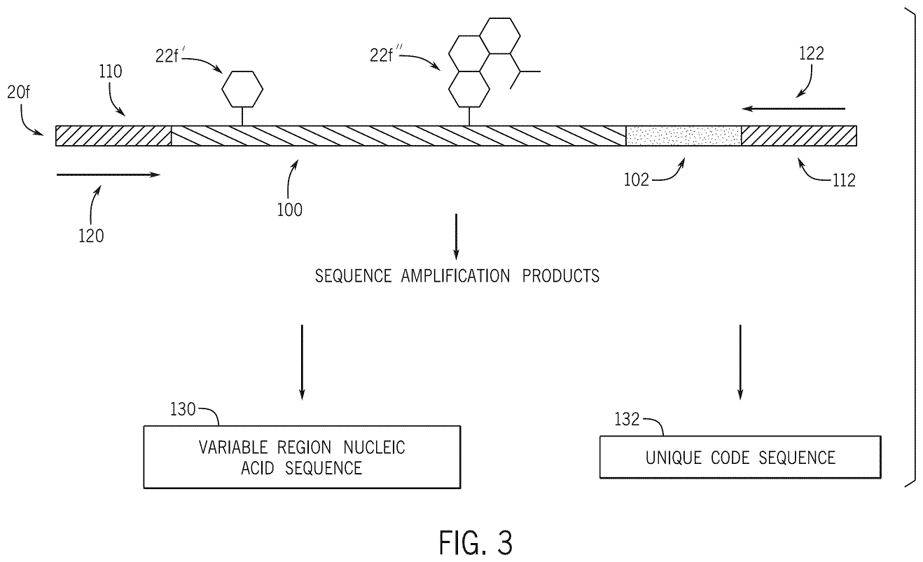

Within each subgroup 14, the aptamer candidates 20 also may generally differ from one another with respect to a variable nucleotide region (see FIG. 3) that may be, in some cases, randomly generated. Accordingly, aptamer candidates 20a may include a mix of different sequences such that aptamer candidates 20a of the subgroup 14a are at least partially different from one another, and so on. Because the individual aptamer candidates 20 within a particular subgroup 14 have at least different nucleotide sequences within a variable region, the incorporation of the modified nucleotide 22 (and any associated molecule) is also variable. That is, if the modified nucleotide 22 is a uridine, one variable sequence of an aptamer candidate 20 may include 10 or more U (e.g., thymidine) sites, while another variable sequence may include 1, 2, 3, or 4 U sites. In addition, these sites can be located at different positions for different aptamer candidates 20. Further, in some cases, the incorporation may not be complete. Thus, if a variable region has 10 available sites, additional diversity may be created by an incomplete incorporation of the modified nucleotide 22 via mixture with an unmodified nucleotide during synthesis. However, a complete incorporation of the modified nucleotide 22 at all available sites may yield more straightforward characterization of candidates at subsequent steps.

Accordingly, although a particular subgroup 14a may be generated using one type of modified nucleotide 22a, or a limited subset of modified nucleotides 22a, the generated aptamer candidates 20a in the subgroup 14a are different from one another at least based on the diversity of variable nucleotide sequences of the aptamer candidates 20a. For example, a first aptamer candidate 20a of the subgroup 14a can include the sequence AAAU*GC as part of a randomly generated variable region, with the modified nucleotide 22a dUTP incorporated at the available fourth position. A second aptamer candidate 20a of the subgroup 14a can include the sequence U*GCGCU* as part of a randomly generated variable region, with the modified nucleotide 22a dUTP incorporated at the available first and sixth positions. It should be understood that these sequences are by way of example. The structure of these different aptamer candidates 20a can be determined based oh sequencing and, in an embodiment, assumption of incorporation of the modified nucleotide 22a at all available sites.

The disclosed embodiments facilitate increase efficiency in candidate selection in a SELEX-type workflow by permitting different types of modifications to be screened together, with aptamer candidates 20 with desired binding characteristics being resolved and characterized through sequencing to determine which, if any, associated nucleotide modifications may have contributed to the binding activity. Thus, rather than screening only one subgroup 14 at a time, a combined library 12 can be screened together. As shown in FIG. 2, aptamer candidates 20 of the library are contacted with a target molecule 30, e.g., a protein. Unbound aptamer candidates 20 can be separated from any bound aptamer candidates 20, and the bound aptamer candidates 20, shown in FIG. 2 as aptamer candidate 20f having modified nucleotide 22f, can be retained. For example, the target molecule 30 may be immobilized on a substrate, and the unbound aptamer candidates 20 can be washed away. Subsequently, the bound aptamer candidate 20 can be separated, e.g., eluted based on a change in buffer conditions, from the target molecule 30 for amplification and subsequent sequencing.

Any nucleotide modifications are not retained after an amplification step. However, by including a unique code for each subgroup 14 that is uniquely associated with a modification type (e.g., the identity and type of the modified nucleotide 22) for the entire subgroup 14, the nucleotide modification information is preserved via the DNA sequence. Thus, different subgroups 14 (e.g., different subgroups 14a, 14b, 14c, 14d, 14c, 14f, 14g) can be distinguished from one another using their unique codes. In the illustrated embodiment, sequencing of the amplified products of any retained candidate aptamers, shown by way of example as aptamer candidate 20f, and identification of both the binding sequence and unique code of the associated modification type, can be used as an input for one or more additional selection cycles. Additional cycles may include negative selection for binding activity to other target molecules. However, as discussed herein, the ability to differentiate between different subgroups 14, and their associated modifications, in a single library 12 via an encoded nucleic acid sequence, permits more diverse aptamer candidates 20 to be screened together in the library 12, thus increasing library selection efficiency and potentially reducing a number of selection cycles.

FIG. 3 shows an example arrangement of the ssDNA aptamer candidate 20f that is retained or selected during the selection cycle of FIG. 2. As noted in FIG. 2, a selected aptamer candidate 20 can be separated from the library 12 based on its binding to the target molecule 30. Any bound aptamer candidates 20 at the separation stage may be uncharacterized. To identify the features that contribute to binding, the retained aptamer candidate 20f is amplified and sequenced.

The aptamer candidate 20f includes a variable region 100 and a code region 102. The variable region 100 and the code region 102 are flanked by primer regions 110, 112. Because the aptamer candidates 20 are at least partially single-stranded, a first primer region 110 can represent a primer binding site that is a reverse complement of a first primer 120, while the second primer region 112 can correspond to the sequence of a second primer 122 that binds to an amplified strand generated from the first primer 120. In an embodiment, the aptamer candidate 20 is between 50 nucleotides and 200 nucleotides in length. The variable region 100 that participates in target binding may be 20-120 nucleotides in length in an embodiment. The code region 102 may be between 5 and 30 nucleotides in length in an embodiment. The primer regions 110, 112 may be between 10 and 30 nucleotides in length in an embodiment. To prevent overlap between a randomly generated variable region 100 and the code region 102, the code region 102 may be formed from only a subset of available nucleotides, e.g., T, C, or A, T, C.

Amplification products are sequenced to generate a variable region nucleic acid sequence 130 and a unique code sequence 132. The unique code sequence 132 can be used to identify the subgroup 14f of the aptamer candidate 20f and its corresponding modified nucleotide type. That is, the code region 102 can be conserved for all members of a subgroup 14 in an embodiment. In the illustrated embodiment, the modified nucleotide 22f may include a mix of different modifications, shown as modified nucleotides 22f′, 22f″. The candidate aptamer 22f, including its modifications, is capable of generating sufficient amplification products, and the presence of the modifications does not prevent amplification. As illustrated, the primer regions 110, 112 are 5′ and 3′ of the variable region 100 and the code region 102. The code region 102 may be 5′ of or 3′ of the variable region 100. Further, as discussed herein, the code region 102 may include two or more different code regions 102, each coding for different aspects of the modification.

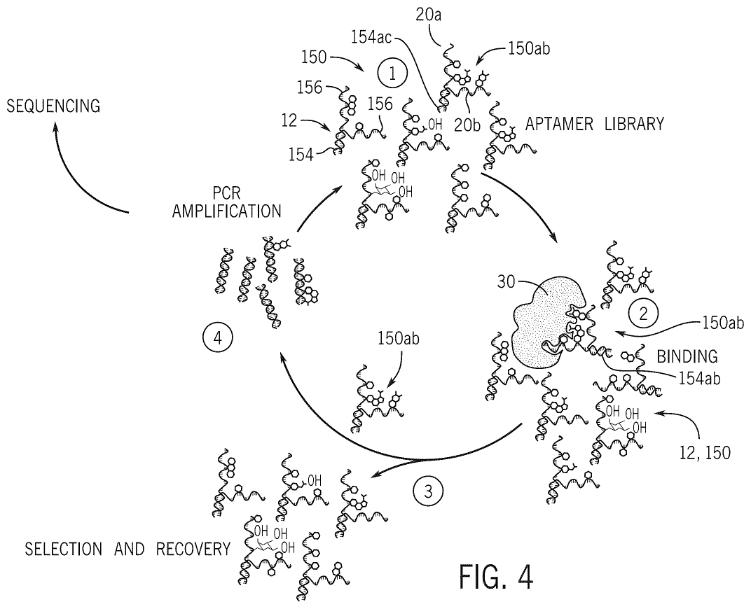

As shown in FIG. 4, an aptamer candidate library 12 can be further expanded in an exponential manner by allowing DNA duplex formation between the aptamer candidates 20. In an embodiment, individual aptamer candidates 20 partially hybridize to one another to form partially duplex aptamer candidates 150. An individual aptamer candidate 20 will be able to be combined with a second aptamer 20 with a different, similar, or same modification, or with no modification at all, rendering a library that is n² more diverse. The advantage that this approach has it that could be extended to more structures displaying more than two variable sites. Further, while the illustrated embodiment shows partially duplex aptamers candidates 150 formed from two different aptamer candidates 20, additional and/or more complex structures may be formed. For example, three, four, five, or more strands of aptamer candidates 20 may be joined in a single structure via complementary region hybridization.

In one embodiment, the partially duplex candidate aptamers 150 are formed from combining different library subgroups (e.g., subgroups 14a, 14b, 14c) with one another and allowing complementary regions on each aptamer candidate 20 to hybridize to form a duplex region 154. Thus, aptamer candidates 20a can, in embodiments, each include a complementary region that is designed to be complementary to a corresponding region on the aptamer candidates 20b, and/or on the aptamer candidates 20c. The complementary region can be designed to avoid self-complementarity to encourage partial duplex region formation between subgroups 14 rather than intrasubgroup combinations. However, it should be understood that intrasubgroup combinations are also encompassed within the disclosure.

In one embodiment, the library 12 can include all or some potential combinations of two or more subgroups 14. In another embodiment, the library 12 can include combinations of one or more subgroups 14 having incorporated modified nucleotides 22 with an unmodified subgroup 14. Further, while the depicted arrangement includes partially duplex candidate aptamers 150 with a terminal duplex region 154 from which respective single-stranded branches 156 extend, other arrangement are also contemplated. In one embodiment, the duplex region 154 is an internal region with single-stranded branches 156 having split variable regions extending in both a 5′ and 3′ direction. Further, one or more of the respective single-stranded branches 156 can be flanked by different duplex regions 154 that link to additional strands.

Once formed, the library 12 can, as generally discussed with respect to FIG. 2, be used to select the partially duplex candidate aptamers 150 with binding activity or specific affinity for a particular target molecule 30 of interest. For example, an individual partially duplex candidate aptamer 150ab, formed from aptamer candidates 20a, 20b that partially hybridize to one another, binds to the target molecule 30 and is retained during selection. As illustrated, the binding activity may be mediated by the single-stranded branches 156, and the duplex region 154ab may not be directly involved in binding. Thus, the region 154ab can be conserved among multiple partially duplex candidate aptamers 150 formed from different aptamer candidates 20a, 20b. After selection, the partially duplex candidate aptamer 150ab is unwound and sequenced.

To retain the identity of the duplex candidate aptamers 150 formed from two different aptamer candidates 20, chemical ligation can be performed to link the component oligonucleotides together, which can then be identified by sequencing the combined construct after PCR amplification. The chemical ligation can be click chemistry ligation or enzyme-mediated ligation.

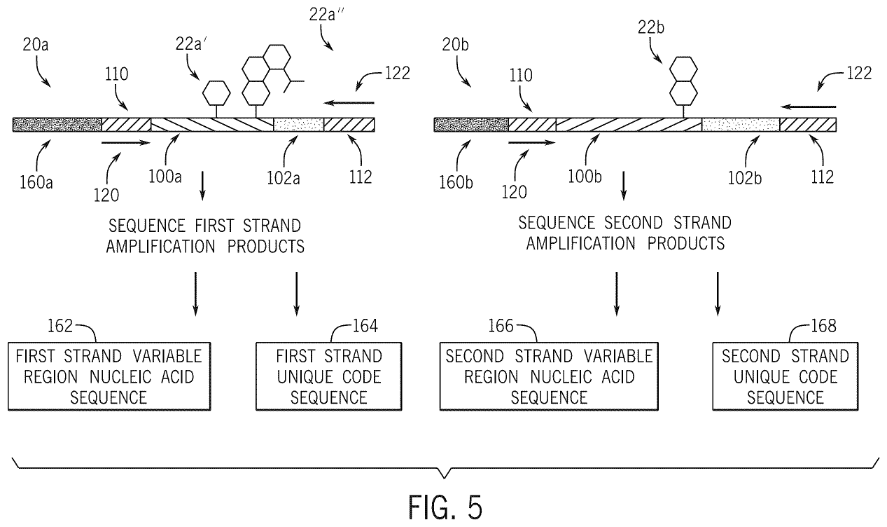

FIG. 5 shows an example arrangement of the partially duplex candidate aptamer 150ab that was retained or selected during the selection cycle of FIG. 4. As noted in FIG. 4, a selected partially duplex candidate aptamer 150ab can be separated from the library 12 based on its binding to the target molecule 30. To identify the features that contribute to binding, the retained partially duplex candidate aptamer 150ab is unwound or denatured to separate the strands of component aptamer candidates 20a, 20b, and the strands of component aptamer candidates 20a, 20b are amplified and sequenced.

The component aptamer candidates 20a, 20b include a variable region 100 and a code region 102 flanked by primer regions 110, 112 as generally discussed with respect to FIG. 3. The primer regions 110, 112 may be conserved between the component aptamer candidates 20a, 20b such that a single set of primers can be used to amplify both strands 20a, 20b or strands of any selected partially duplex candidate aptamer 150. In addition, the component aptamer candidates 20 used for partial duplex formation also include complementary regions 160 that hybridize to one another, e.g., are complementary to one another. In an embodiment, the complementary regions are positioned outside of the amplified areas such that only the active, single-stranded portions of the aptamer candidates 20 are amplified for efficiency. However, other arrangements are also contemplated, and one or both of the primer regions 110, 112 may be part of a complementary region 160. In the partially duplex candidate aptamer 150ab, the variable region 100a of the first strand aptamer 20a may be different than the variable region 100b of the second strand aptamer 20b. Further, the respective codes regions 102a, 102b are different, reflective different modification types. Finally, the complementary regions 160a, 160b are reverse complements to facilitate partial duplex formation.

The amplification products of both component aptamer candidates 20a, 20b are both sequenced to generate a first strand variable region nucleic acid sequence 162, a first strand unique code sequence 164, a second strand variable region nucleic acid sequence 162, and a second strand unique code sequence 168. The unique code sequences 164, 168 can be used to identify the subgroup 14a, 14b of the aptamer candidates 20a, 20b and corresponding modified nucleotide types, shown as 22a′a″ and 22b. That is, the code region 102 can be conserved for all members of a subgroup 14 in an embodiment.

FIG. 6 shows functionalities that can be added to modified nucleotides 22. It should be understood that these are by way of example, and other modifications are also contemplated to be within the scope of the disclosed embodiments. For example, the modified nucleotide 22 may include a phosphate modifications: methylphosphonate (neutral) phosphorothioate (anionic) guanidinopropyl phosphoramidate (cationic). Additionally or alternatively, the modified nucleotide 22 may include a sugar modifications: 2′-F; 2′-amino; 2′-OMe; 2′-azido; Conformationally Locked Sugar (LNA) X=O, LNA; X=NR, amino-LNA; X=S, thio-LNA. Additionally or alternatively, the modified nucleotide 22 may include a phosphate replacement: triazole (neutral); guanidinium (cationic). Additionally or alternatively, the modified nucleotide 22 may include a purine modifications: 2,6-diaminopurine; 3-deaza-adenine; 7-deaza-guanine; 8-azido-adenine. Additionally or alternatively, the modified nucleotide 22 may include a pyrimidine modification: 2-thio-thymidine; 5-carboxamide-uracil 5-methyl-cytosine; 5-ethynyl-uracil (CLICK chemistry site).

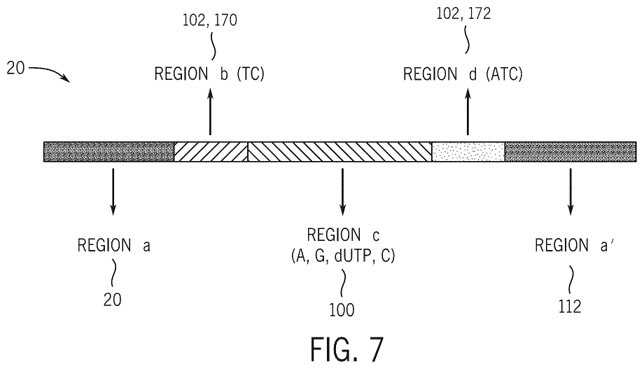

As provided herein, the modified nucleotide can include non-standard functionalities incorporated by click chemistry by introducing an azide modification. This non-standard functionality has the following characteristics: based on a fragment small molecule, e.g., a fragment known to bind weakly to the target protein, and its position is encoded in the DNA sequence in a binary code based on T(0), C(1) to avoid G-quadruplexes as generally discussed with respect to FIG. 7.

In one embodiment, the modified nucleotide can include a modification with almost any azide-bearing functional group that can be conjugated to an alkyne-modified (at C5 site) dU via facile copper-catalyzed azide-alkyne cycloaddition (CuAAC) or “click chemistry”. In an embodiment, the modification can be generated based on a starting 5-Ethynyl uridine or 5-Ethynyl uracil (Jena Bioscience) that includes an alkyne that can be subsequently ligated to azide containing molecules through click ligation.

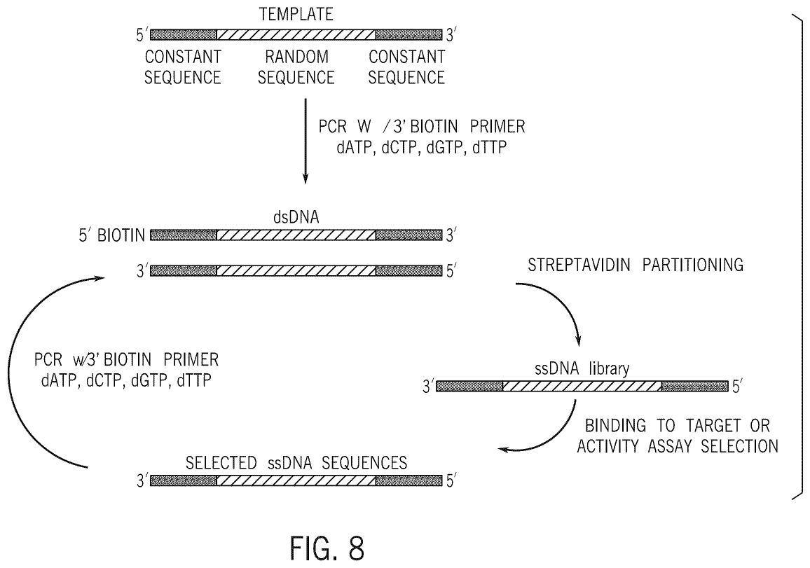

Primer extension was shown to work on modified templates to give good to excellent yields of full-length DNA. Therefore it is possible to use selection rounds as in traditional SELEX (see FIG. 8). In addition, an adapted SELEX approach that involves the combination of ssDNA into duplexes can also be implemented (see FIG. 4) further increasing the chemical space of the SELEX library.

As provided herein, aptamer candidate techniques can combine two molecular evolution technologies used for finding binding molecules that have high specificity and selectivity to a target molecule of interest by merging of DNA-encoded small organic ligands and ssDNA SELEX. At step one, synthesis and design of the hybrid small molecule-DNA library and the selection of the target molecule is performed. Standard methods employing phosphoramidite chemistry will be used for the synthesis of the hybrid small molecule-DNA library. The attachment of small organic molecules or fragments will be explored using two strategies. The first approach makes use of the post modification clickSELEX. In this approach chemical groups are introduced into the DNA library before the selection step via click chemistry and are subsequently removed during the amplification step. By doing so enzymatic incompatibility problems associated with larger nucleobase modifications are avoided. Hence, the disclosed embodiments allow for an easy implementation of a multitude of different chemical functionalities adapted to the imposed requirements. Mayer at al. have reported a rapid and cost-effective protocol for the high-fidelity largescale generation of nucleobase-modified nucleic acids. The solid phase synthesis of EdU containing DNA, will be followed by a click-reaction with the DNA still attached to the solid phase and subsequent deprotection and purification according to the standard procedures. As a linker for the ssDNA and the small molecules 5-ethynyl-2′-deoxyuridine (EdU) will be used.

FIG. 7 shows an example aptamer candidate 20. The location of the “clickable” nucleotide is encoded in the DNA sequence using a binary code base on TC bases to avoid G-quadruplexes. The design of the library members will be as follows; regions a and a′ correspond to conserved primer sequences 110, 112 for PCR amplification. Region b corresponds to a first portion 170 of a code region 102 with a unique short DNA sequence of 6 nucleotides (TC coded) to identify the location in region c (variable region 100) with the clickable nucleotide, e.g., the modified nucleotide 22, that will be attached to a molecular fragment-“small molecules”. Region c can be a random sequence of 40-50 nucleotides including the non-clickable and clickable functionalized nucleotides. Region d corresponds to a second portion 172 of the code region 102 that contains the code for the identity of the aromatic non-clickable and the clickable nucleotides (coded as ATCG bases).

FIG. 8 shows an example selection cycle that begins with the chemical synthesis of a template DNA strand library (ca. 1014 sequences) composed of a random sequence flanked by fixed sequence regions required for PCR. PCR amplification of this template library in the presence of a 3′-primer containing a biotin capture molecule at its 5′-end yields biotinylated dsDNA that can be captured using a reaction with streptavidin. Note that this step helps ensure that the sequences to go forward into selection are amplifiable. The dsDNA is then treated with streptavidin immobilized on beads, and the complement strand is removed from the beads by heating or raising the pH to generate a ssDNA library in solution. CE-SELEX works in a different approach, without the need of biotin-functionalized primer.

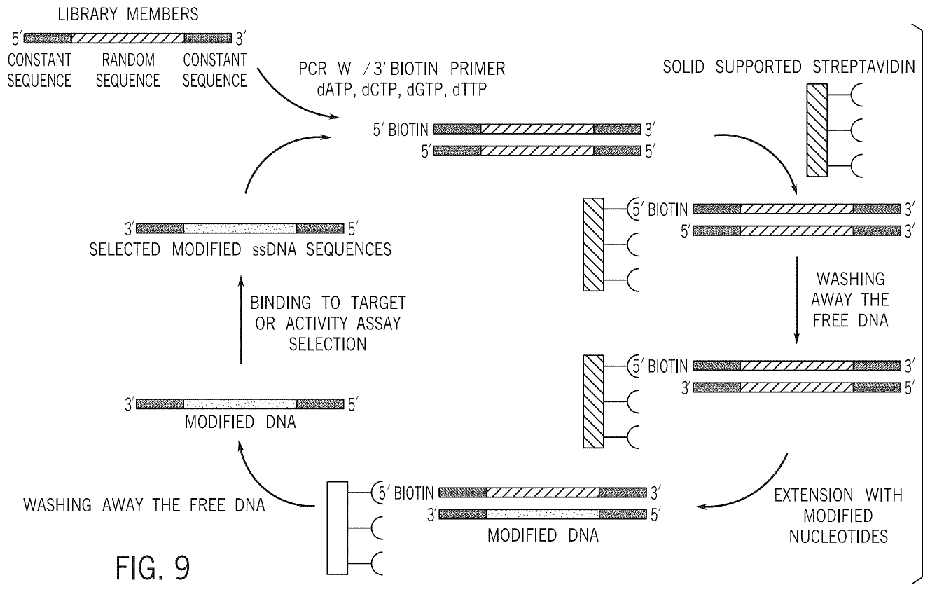

FIG. 9 shows a selection cycle that begins with a DNA template library prepared by automated DNA synthesis. The amide linkage of the modified dUTP derivatives is stable to basic conditions for extended periods of time at ambient temperature. This modified ssDNA library could then undergo the selection step for binding of the protein of interest or any other target. The selected sequences would then go into PCR with TTP, dATP, dCTP, and dGTP, avoiding PCR amplification using the modified dUTP. Using the same 3′-primer containing a 5′-biotin would create an enriched library ready to begin the next cycle of selection. An additional next generation sequencing (NGS) step at the end of the selection may be performed when adding extra small molecular fragments.

The small fragments can be selected so they can contribute to the binding affinity between aptamers and their targets by hydrogen bonding, structure compatibility, stacking of aromatic rings, electrostatic and hydrophobic interactions and van der Waals forces. To this end the first choice is to add extra-small molecules as fragments. The small molecules will be selected taking into consideration their commercial availability and hydrophobicity. The library will consist of 1000 different fragments combined with the nucleotide random sequences that contain the modified base dUTP resulting into a hybrid library

At step two, optimal conditions for amplification of the library by PCR are identified. Positive and negative rounds of selection will be used for the identification of the hybrid aptamers. High-Throughput Sequencing SELEX (HTS-SELEX) will be used since allows for sequencing of the library across all the selection rounds. The identification of the DNA encoded-molecular fragments will be done in each round and thus enriched sequences will be visible at a much earlier stage, making the process more time efficient. The synthetic alkyne-modified DNA library is functionalized with an azide fragment by CuAAC-click chemistry. After selection and the removal of unbound library members, the bound sequences are isolated and amplified by PCR employing the alkyne-modified triphosphate instead of thymidine (alternatively the azide modification can also be used). Therefore, the modification in the elongating strand is removed, and the alkyne moiety is reintroduced. After PCR, the single stranded DNA is prepared by A-exonuclease digestion of 5′-phosphorylated antisense strands. The modification is then reintroduced by CuAAC-click chemistry, and the obtained library is used for the next SELEX cycle.

At step three, identification of the best-binding molecule is performed. Standard NGS sequencing techniques will be used to determine the DNA sequence of the aptamers. The identity of the small molecule fragment will be revealed by its unique DNA code. Since HTS-SELEX will be used for each of the selection rounds it is expected an early recognition of the best binding aptamers.

At step four, characterization of the binding properties of the hybrid aptamers is performed. Their binding affinity of the selected aptamers will be measured using ITC (Isothermal calorimetry), fluorescent polarization spectroscopy and SPR (Surface Plasmon Resonance). ITC measurements will give information related to the thermodynamics of the binding event between the ligand and the target molecule. SPR will be used to determine the kinetics of the binding event (Kon/Koff). Finally, fluorescent polarization spectroscopy can be performed by the introduction of a fluorophore in the aptamer structure.

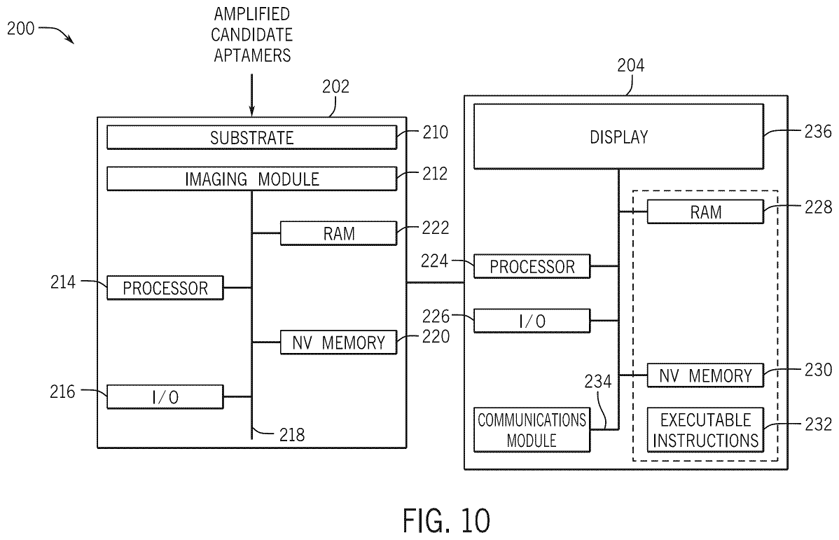

In some embodiments, the disclosed techniques are used to generate sequence data from amplified aptamer candidates 20. FIG. 10 is a schematic diagram of a sequencing device 200 that may be used in conjunction with the disclosed embodiments for acquiring sequencing data from candidate aptamers as provided herein. The sequence device 200 may be implemented according to any sequencing technique, such as those incorporating sequencing-by-synthesis methods described in U.S. Patent Publication Nos. 2007/0166705; 2006/0188901; 2006/0240439; 2006/0281109; 2005/0100900; U.S. Pat. No. 7,057,026; WO 05/065814; WO 06/064199; WO 07/010,251, the disclosures of which are incorporated herein by reference in their entireties. Alternatively, sequencing by ligation techniques may be used in the sequencing device 200. Such techniques use DNA ligase to incorporate oligonucleotides and identify the incorporation of such oligonucleotides and are described in U.S. Pat. Nos. 6,969,488; 6,172,218; and 6,306,597; the disclosures of which are incorporated herein by reference in their entireties.

In the depicted embodiment, the sequencing device 200 includes a separate sample substrate 202, e.g., a flow cell or sequencing cartridge, and an associated computer 204. However, as noted, these may be implemented as a single device. In the depicted embodiment, the biological sample may be loaded into substrate 210 that is imaged to generate sequence data. For example, reagents that interact with the biological sample fluoresce at particular wavelengths in response to an excitation beam generated by an imaging module 212 and thereby return radiation for imaging. For instance, the fluorescent components may be generated by fluorescently tagged nucleic acids that hybridize to complementary molecules of the components or to fluorescently tagged nucleotides that are incorporated into an oligonucleotide using a polymerase. As will be appreciated by those skilled in the art, the wavelength at which the dyes of the sample are excited and the wavelength at which they fluoresce will depend upon the absorption and emission spectra of the specific dyes. Such returned radiation may propagate back through the directing optics. This retrobeam may generally be directed toward detection optics of the imaging module 212, which may be a camera or other optical detector.

The imaging module detection optics may be based upon any suitable technology, and may be, for example, a charged coupled device (CCD) sensor that generates pixilated image data based upon photons impacting locations in the device. However, it will be understood that any of a variety of other detectors may also be used including, but not limited to, a detector array configured for time delay integration (TDI) operation, a complementary metal oxide semiconductor (CMOS) detector, an avalanche photodiode (APD) detector, a Geiger-mode photon counter, or any other suitable detector. TDI mode detection can be coupled with line scanning as described in U.S. Pat. No. 7,329,860, which is incorporated herein by reference. Other useful detectors are described, for example, in the references provided previously herein in the context of various nucleic acid sequencing methodologies.

The imaging module 212 may be under processor control, e.g., via a processor 214, and may also include I/O controls 216, an internal bus 218, non-volatile memory 220, RAM 222 and any other memory structure such that the memory is capable of storing executable instructions, and other suitable hardware components that may be similar to those described with regard to FIG. 10. Further, the associated computer 204 may also include a processor 224, I/O controls 226, a communications module 234, and a memory architecture including RAM 228 and non-volatile memory 230, such that the memory architecture is capable of storing executable instructions 232. The hardware components may be linked by an internal bus 194, which may also link to the display 236. In embodiments in which the sequencing device 200 is implemented as an all-in-one device, certain redundant hardware elements may be eliminated.

The processor 214 may be programmed to assign individual sequencing reads to a subgroup 14 based on the associated unique code sequence or sequences according to the techniques provided herein. Each sequence read may include both the variable region nucleic acid sequence and the unique code sequence. The sequencing data includes base calls for each base of a sequencing read.

As used herein, an aptamer may refer to a non-naturally occurring nucleic acid that has specific binding affinity for a target molecule. In certain embodiments, aptamer candidates 20 are provided which are nucleic acids of unknown binding ability that may, upon screening and selection, be determined to have sufficient binding affinity to be selected as aptamers for a particular application. In certain cases, aptamer candidate 20 and aptamer may be used interchangeably in the disclosure. The binding of the aptamer to the target molecule can result in catalytically changing the target molecule, reacting with the target molecule in a way that modifies or alters the target molecule or the functional activity of the target molecule, covalently attaching to the target molecule (as in a suicide inhibitor), and facilitating the reaction between the target molecule and another molecule. In one embodiment, the target molecule is a three dimensional chemical structure, other than a polynucleotide, that binds to the aptamer through a mechanism which is predominantly independent of Watson/Crick base pairing or triple helix binding. In an embodiment, the aptamer is not a nucleic acid having the known physiological function of being bound by the target molecule.

Aptamers include nucleic acids that are identified from a candidate mixture of nucleic acids (e.g., aptamer candidates). Further, a pool of aptamer candidates can include one or more aptamers of interest for a particular target molecule. The aptamer can be identified from the aptamer candidates as being a ligand of a given target molecule by contacting the candidate mixture with the target, wherein nucleic acids having an increased affinity to the target relative to other nucleic acids in the candidate mixture may be partitioned from the remainder of the candidate mixture. A specific binding affinity of an aptamer for its target may refer to aptamer binding to its target generally with a much higher degree of affinity than it binds to other, non-target, components in a mixture or sample. Different aptamers may have either the same number or a different number of nucleotides. Aptamers may be DNA or RNA and may be single stranded, double stranded, or contain double stranded regions.

The disclosed aptamers and/or aptamer candidates can be used to generate an aptamer capable of modifying the bioactivity of a target through binding and/or crosslinking to the target. In one embodiment, an aptamer to a unique target associated with or relevant to a specific disease process is identified. This aptamer can be used as a diagnostic reagent, either in vitro or in vivo. In another embodiment, an aptamer to a target associated with a disease state may be administered to an individual and used to treat the disease in vivo. The aptamers identified herein can be used in any diagnostic, imaging, high throughput screening or target validation techniques or procedures or assays for which aptamers, oligonucleotides, antibodies and ligands, without limitation can be used.

In certain embodiments of the disclosure, aptamer candidates 20 can include one or more conserved regions, such as a conserved primer region, e.g., a first conserved primer region and a second conserved primer region. A conserved region is conserved between at least some other aptamer candidates 20 such that the conserved region has an identical or similar nucleotide sequence as compared between the aptamer candidates 20. In an embodiment, a conserved region is conserved in both nucleotide sequence and position between different aptamer candidates 20. In an embodiment, the conserved region has an identical sequence such that two or more different aptamer candidates 20 have a same sequence in the conserved region. In an embodiment, a conserved region has fewer than two different nucleotides between different aptamer candidates 20 having the conserved region. In an embodiment, the conserved region may be a universal region. For example, for a given library 12, all aptamer candidates in the library 12 can have a same first conserved primer region and a second conserved primer region. In this manner, primers based on the first conserved primer region and the second conserved primer region can be used to amplify any selected aptamer candidate 20 of the library 12. In an embodiment, the conserved region may be conserved only within a particular subgroup 14, such that aptamer candidates 20 of a particular subgroup 14 all have a conserved code region or conserved complementary region, but other aptamer candidates 20 of different subgroups 14 have a different code region and/or complementary region.

A conserved primer region can include a region having the sequence of a universal Illumina® capture primer or a region specifically hybridizing with a universal Illumina® capture primer. Universal Illumina® capture primers include, e.g., P5 5′-AATGATACGGCGACCACCGA-3′ ((SEQ ID NO: 1)) or P7 (5′-CAAGCAGAAGACGGCATACGA-3′ (SEQ ID NO: 2)), or fragments thereof. A region specifically hybridizing with a universal Illumina® capture primer can include, e.g., the reverse complement sequence of the Illumina® capture primer P5 (“anti-P5”: 5′-TCGGTGGTCGCCGTATCATT-3′ (SEQ ID NO: 3) or P7 (“anti-P7”: 5′-TCGTATGCCGTCTTCTGCTTG-3′ (SEQ ID NO: 4)), or fragments thereof.

A conserved primer region can additionally or alternatively include a region having the sequence of an Illumina® sequencing primer, or fragment thereof, or a region specifically hybridizing with an Illumina® sequencing primer, or fragment thereof. Illumina® sequencing primers include, e.g., SBS3 (5′-ACACTCTTTCCCTACACGACGCTCTTCCGATCT-3′ (SEQ ID NO: 5)) or SBS8 (5′-CGGTCTCGGCATTCCTGCTGAACCGCTCTTCCGATCT-3′ (SEQ ID NO: 6)). A region specifically hybridizing with an Illumina® sequencing primer, or fragment thereof, can include, e.g., the reverse complement sequence of the Illumina® sequencing primer SBS3 (“anti-SBS3”: 5′-AGATCGGAAGAGCGTCGTGTAGGGAAAGAGTGT-3′ (SEQ ID NO: 7)) or SBS8 (“anti-SBS8”:

5′-AGATCGGAAGAGCGGTTCAGCAGGAATGCCGAGACCG-3′ (SEQ ID NO: 8)), or fragments thereof. The incorporation of sequencing primer sequences in the candidate aptamers may be either directly or via subsequent amplification, ligation, or other sequencing library preparation steps.

This written description uses examples to enable any person skilled in the art to practice the disclosed embodiments, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An aptamer candidate library, comprising:
a plurality of at least partially single-stranded nucleic acids, wherein an individual at least partially single-stranded nucleic acid of the plurality comprises:
a first conserved primer region that is conserved among the plurality of at least partially single-stranded nucleic acids;
a second conserved primer region that is conserved among the plurality of at least partially single-stranded nucleic acids;
a variable region disposed between the first conserved primer region and the second conserved primer region, wherein the variable region is variable among the plurality of at least partially single-stranded nucleic acids and wherein each variable region comprises at least one modified nucleotide; and
a code region, wherein the code region comprises a nucleotide sequence that is unique for a modification type of the at least one modified nucleotide.

2. The library of claim 1, wherein the plurality of at least partially single-stranded nucleic acids comprises multiple modification types, each corresponding to a different code region having a respective different nucleotide sequence.

3. The library of claim 2, wherein the variable region of the individual at least partially single-stranded nucleic acid of the plurality comprises only a single modification type of the at least one modified nucleotide.

4. The library of claim 3, wherein the variable region comprises two of more of the at least one modified nucleotide having the single modification type of the at least one modified nucleotide.

5. The library of claim 1, wherein the modification type comprises modification with a molecular fragment.

6. The library of claim 1, wherein the modification type comprises a chemically modified uridine.

7. The library of claim 1, wherein the modification type comprises modification via a click chemistry reaction.

8. The library of claim 7, wherein the at least one modified nucleotide is modified with an alkyne.

9. The library of claim 1, wherein the individual at least partially single-stranded nucleic acid of the plurality further comprises: a complementary region that forms a duplex region with another at least partially single-stranded nucleic acid of the plurality.

10. The library of claim 9, wherein the complementary region is not between the first conserved primer region and the second conserved primer region.

11. The library of claim 1, wherein the code region is positioned between the first conserved primer region and the second conserved primer region.

12. The library of claim 1, wherein the code region comprises a first portion identifying a modification of the modified nucleotide and a second portion identifying a location within the variable region of the modified nucleotide.

13. The library of claim 12, wherein the second portion comprises a code sequence encoded with only two different nucleotides.

14. The library of claim 12, wherein the first portion and the second portion flank the variable region and are between the first conserved primer region and the second conserved primer region.

15. An aptamer selection method, comprising:
providing a library of aptamer candidates of different aptamer subgroups, wherein each aptamer candidate of an individual subgroup of the different aptamer subgroups comprises:
a first conserved primer region that is conserved among the aptamer candidates;
a second conserved primer region that is conserved among the aptamer candidates;
a variable region disposed between the first conserved primer region and the second conserved primer region, wherein the variable region is variable among the aptamer candidates and wherein the variable region comprises at least one modified nucleotide; and
a code region, wherein the code region comprises a nucleotide sequence that is unique for a modification type of the at least one modified nucleotide and that uniquely identifies the modification type from different modification types of other subgroups of the different subgroups;
selecting an aptamer candidate based on binding to a target molecule;
amplifying the selected aptamer candidate using primers based on the first and second conserved primer region; and
sequencing the amplified aptamer candidate to determine a sequence of the variable region and to identify the modification type.

16. The method of claim 15, wherein the code region is conserved within the individual subgroup such that aptamer candidates of the individual subgroup all have a same code region.

17. The method of claim 15, comprising separating amplified aptamer candidates using a capture molecule.

18. An aptamer candidate library, comprising:
a plurality of partial duplex aptamer candidates comprising a first strand and a second strand, the first strand comprising:
a duplex portion comprising a first complementary region that hybridizes to a second complementary region of the second strand;
a single-stranded portion, the single-stranded portion comprising:
a first conserved primer region that is conserved among first strands and second strands of the plurality of partial duplex aptamer candidates;
a second conserved primer region that is conserved among the first strands and the second strands of the plurality of partial duplex aptamer candidates;
a variable region disposed between the first conserved primer region and the second conserved primer region, wherein the variable region is variable among the first strands and the second strands of the plurality of partial duplex aptamer candidates and that comprises at least one modified nucleotide; and
a code region, wherein the code region comprises a nucleotide sequence that is unique for a modification type of the at least one modified nucleotide.

19. The library of claim 18, wherein the second strand comprises:
a second strand single-stranded portion, the second strand single-stranded portion comprising the first conserved primer region and the second conserved primer region;
a second strand variable region different than the variable region of the first strand; and
a second strand code region, different than the code region of the second strand.

20. The library of claim 19, wherein the second strand comprises a second modification type different than the modification type of the first strand.
